(19) United States
(12) Patent Application Publication
(10) Pub. No.: US 2025/0104393 A1
(43) Pub. Date: Mar. 27, 2025

(54) Personal Profile Generator and Recommendation Engine

(71) Applicant: Johnson & Johnson Consumer Inc., Skillman, NJ (US)

(72) Inventors: Nikoleta N. Batchvarova, West Windsor, NJ (US); Desiree Dowe, Playa Del Rey, CA (US); Ralph Charles Evert, IV, Doylestown, PA (US); Russell Gould, Washington Crossing, PA (US); Diana E. Gutierrez Lopez, Cranford, NJ (US); Logan McGill, Philadelphia, PA (US); Andrew Amirov, Fair Lawn, NJ (US); Amelia Paladino, Hoboken, NJ (US); Shane M. Tomasello, Voorhees, NJ (US); Lia X. Al-Ghalith, Waltham, MA (US)

(21) Appl. No.: 18/813,894

(22) Filed: Aug. 23, 2024

Related U.S. Application Data
(60) Provisional application No. 63/539,865, filed on Sep. 22, 2023.

(51) Int. Cl. G06V 10/762; G06T 7/00; G16H 20/10

(52) U.S. Cl. CPC G06V 10/762; G06T 7/0012; G16H 20/10; G06T 2207/30201

(57) ABSTRACT

Systems and methods for generating a personal profile and a recommendation based on the generated personal profile. The method includes generating a plurality of clusters, each cluster of the generated plurality of clusters including at least a set of variables for users included in the cluster, the set of variables related to at least one of gender, age, skin tone, acne marks, acne frequency, a lesion score, or a body distribution score, generating, for a new user, a profile, associating the generated profile into a cluster of the plurality of clusters, and generating, by a machine learning (ML) model, a recommendation for the user based on the associated cluster.

### CROSS-REFERENCE TO RELATED APPLICATION

This application claims priority under 35 U.S.C. § 119 (e) to U.S. Provisional Application No. 63/593,865 filed on Sep. 22, 2023, the contents of which is incorporated herein by reference in its entirety.

### BACKGROUND

Traditionally, consumers of healthcare products select a particular product that treats a particular condition. The condition may be a skin condition, such as acne or dandruff, a hair condition, a phase of life such as menopause, skin aging, and so forth. Oftentimes, the condition is self-diagnosed by the consumer and the particular healthcare product is selected based on an advertisement, word of mouth, or selection in store or online. However, the self-diagnosis may not be correct and/or may not take into account the root cause of a particular symptom or symptoms, and the selected product may not be the most effective product for the root cause of the symptom.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one example, a computer-implemented method is provided. The method includes generating a plurality of clusters; generating, for a new user, a profile; associating the generated profile into a cluster of the plurality of clusters; and generating, by a machine learning (ML) model, a recommendation for the user based on the associated cluster.

In another example, an apparatus is provided. The apparatus includes a user interface (UI); a memory; an image capturing device configured to capture a facial scan of a user; and a processor coupled to the memory configured to: control the UI to present a questionnaire; receive, via the UI, a response to the questionnaire; generate a profile associated with the user based on the received responses to the questionnaire and the captured facial scan; associate the generated profile into a cluster of a plurality of clusters; and execute, a machine learning (ML) model to generate a recommendation for the user based on the associated cluster.

In another example, a computer-readable storage media is provided. The computer-readable storage media stores instructions that, when executed by a processor, cause the processor to generate a plurality of clusters, each cluster of the generated plurality of clusters including at least a set of variables for users included in the cluster, the set related to at least one of gender, age, skin tone, acne marks, acne frequency, a lesion score, or a body distribution score; generate, for a new user, a profile, the generated profile including at least one variable of the set of variables; associate the generated profile into a cluster of the plurality of clusters; and executing a machine learning (ML) model to generate a recommendation for the user based on the associated cluster.

Other technical features may be readily apparent to one skilled in the art from the following figures, descriptions, and claims.

### BRIEF DESCRIPTION OF THE DRAWINGS

The present description will be better understood from the following detailed description read in light of the accompanying drawings, wherein:

FIG. 1 illustrates an example system for generating a personal profile and a recommendation based on the generated personal profile;

FIG. 2 illustrates an example system for generating a plurality of personas;

FIG. 3 illustrates an example of a recommendation engine for generating interventions for a particular profile;

FIG. 4 illustrates an example timeline of a profile updating over time;

FIG. 5 illustrates an example health outcome over time;

FIG. 6 illustrates an example for building profiles and associated clusters for one or more example profiles;

FIGS. 7A-7F illustrate example user interfaces (UIs) of a flow including creating a profile, performing a facial scan, receiving data, and generating a recommendation;

FIG. 8 illustrates an example computer-implemented method of generating one or more recommendations for a profile; and

FIG. 9 is a block diagram illustrating an example computing environment suitable for implementing one or more of the various examples disclosed herein.

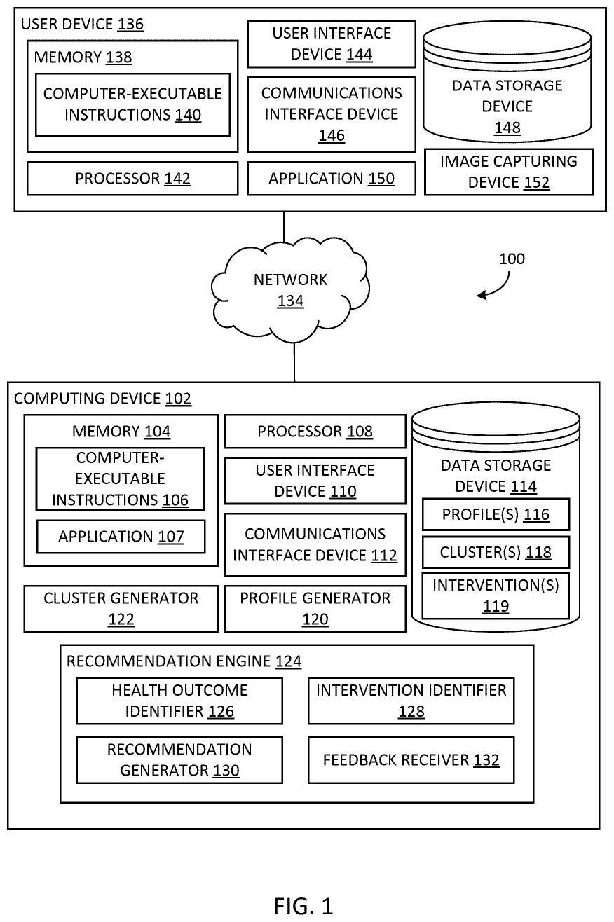

Corresponding reference characters indicate corresponding parts throughout the drawings. In FIGS. 1 to 9, the systems are illustrated as schematic drawings. The drawings may not be to scale.

### DETAILED DESCRIPTION

The various implementations and examples will be described in detail with reference to the accompanying drawings. Wherever possible, the same reference numbers will be used throughout the drawings to refer to the same or like parts. References made throughout this disclosure relating to specific examples and implementations are provided solely for illustrative purposes but, unless indicated to the contrary, are not meant to limit all examples.

As described herein, identification of a particular condition, such as acne, skin aging, or dandruff, is traditionally performed by a consumer via self-diagnosis, which may fail to take into account an underlying cause of the symptom. Moreover, treatment typically involves treatment of the condition without addressing the underlying cause of the symptom. For example, a consumer may identify a condition, such as acne, and select a particular product that treats acne without a full understanding of why the acne presents, the most effective product to treat the acne, or other or different products that treat an underlying cause of the acne. Moreover, over time a consumer may default to the selection of a previously selected product that was effective at a previous time, but due to changes in the consumer's age, lifestyle, environment, and so forth may no longer be the most effective product to treat the same condition. Current solutions fail to adequately take into account underlying causes of various symptoms in order to recommend an optimal product or lifestyle adjustment, as well as fail to address evolving aspects of a consumer's profile.

Accordingly, aspects of the present disclosure provide systems and methods for generating a personal profile for a consumer, identifying one or more underlying causes of one or more conditions, and generating, using artificial intelligence (AI), holistic product and/or lifestyle recommendations to address the one or more conditions. In some examples, the recommendations are updated over time to incorporate feedback regarding the implementation of a recommendation, a change in the condition or the profile of the consumer, and so forth. The systems and methods described herein operate in an unconventional manner by collecting data from various sources and in various formats for a new profile, converting the collected data into a standardized format, generating clusters of similar profiles and associating the new profile with a particular cluster, determining an expected health outcome for the profiles in the cluster and identifying an associated intervention, and generating a recommendation that, when applied, implements the intervention to address the expected health outcome. The present disclosure therefore provides numerous technical effects, including an improved data structure that stores the converted data originally collected from multiple sources that facilitates improved retrieval of the information, an improved recommendation engine that implements a trained machine learning model to optimizes the content delivered, i.e., the generated recommendation, to a consumer based on specific historical user characteristics, taking into account changes in the consumer's profile over time.

For example, the present disclosure provides a profile generator that generates a profile for a consumer. The profile includes variables related to consumer data including, but not limited to, gender, age, a lesion score, a skin tone, whether the consumer has acne marks/scars, a frequency of acne, and a body distribution score. A cluster generator generates a cluster of similar profiles. Similar profiles are profiles that have a determined similarity to be above a similarity threshold based on the values of the variables. A recommendation engine generates a health outcome for a particular cluster, identifies an intervention for the health outcome, and generates a recommendation including the intervention. The recommendation is provided to the consumer. In some examples, the recommendation engine receives feedback regarding the provided recommendation and is updated, based on the received feedback, to improve further recommendations. In some examples, the cluster into which a particular profile is associated changes over time. For example, as a consumer's age, lesion score, acne frequency, or body distribution score changes, such as based on the implementation of a recommended intervention, the profile is more closely associated with a different cluster. The recommendation engine then generates an updated recommendation based on the new cluster.

Various examples described here provide a proactive application, or kit, to deliver physical interventions that will change health outcomes. In some examples, the application is an aspect of one or more bundled products that includes the application, content, and a gamified journey point system.

In some examples, the cluster a particular profile is associated with changes over time. For example, as a consumer's age changes, recommendations are implemented that change the profile of the consumer, and so forth, the profile transitions from one cluster to another over time. This is referred to as a personal trajectory. Accordingly, aspects of the present disclosure enable a journey for a consumer to identify causes of health outcomes, such as acne, gut health, etc., and the reasoning behind those causes, as well as what traits and characteristics a consumer may be prone to, and provides recommendations that include interventions to understand how to address and mitigate negative traits and characteristics and enhance positive traits and characteristics.

As described herein, various examples of the present application provide a technical solution to the inherently technical problem of identifying similarities between digital profiles and associating a newly generated profile with a group of existing profiles based at least in part on digital image analysis, and further detecting changes to a profile over time in order to maintain association of the profile with the closest group of existing profiles. This solution provides at least two technical advantages, including reducing the consumption of computing resources over existing solutions that begin a new categorization process of a user each time an update is made to a user's profile, and generating and implementing an improved set of indexes to facilitate the retrieval of the profile and/or cluster information of a user for the generation of a user recommendation.

FIG. 1 an example system for generating a personal profile and a recommendation based on the generated personal profile. The system 100 illustrated in FIG. 1 is provided for illustration only. Other examples of the system 100 can be used without departing from the scope of the present disclosure. In some examples, the system 100 generates a personal profile and recommendation based on a user having a condition, such as acne. In some examples, the system 100 generates a personal profile and recommendation based on a user experiencing a particular phase of life, such as menopause.

The system 100 includes a computing device 102, a network 134, and a user device 136. The computing device 102 represents any device executing computer-executable instructions 106 (e.g., as application programs, operating system functionality, or both) to implement the operations and functionality associated with the computing device 102. The computing device 102 in some examples includes a mobile computing device or any other portable device. A mobile computing device includes, for example but without limitation, a mobile telephone, laptop, tablet, computing pad, netbook, gaming device, and/or portable media player. The computing device 102 can also include less-portable devices such as servers, desktop personal computers, kiosks, or tabletop devices. Additionally, the computing device 102 can represent a group of processing units or other computing devices.

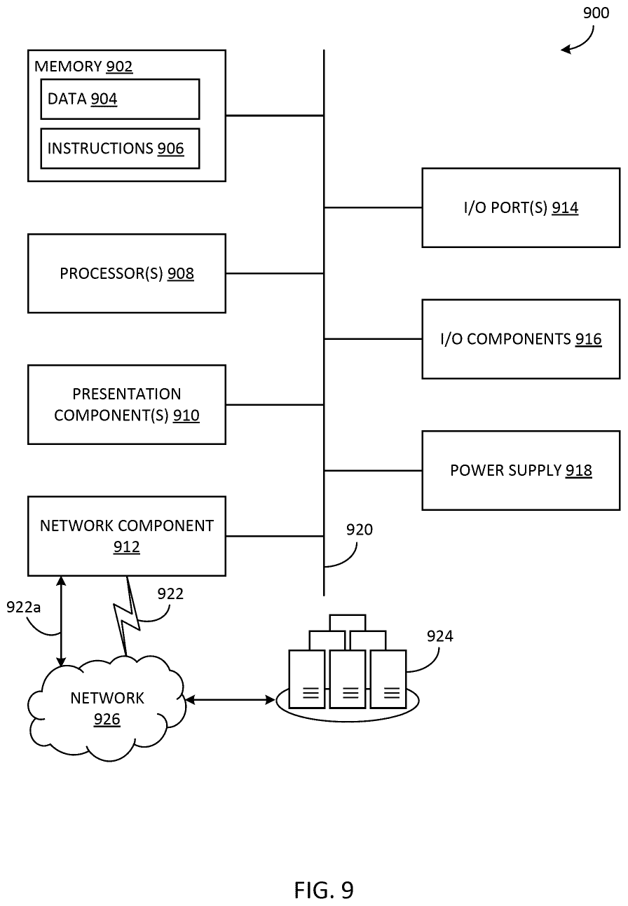

In some examples, the computing device 102 includes at least one processor 108, a memory 104 that includes the computer-executable instructions 106, and a user interface device 110. The processor 108 includes any quantity of processing units and is programmed to execute the computer-executable instructions 106. The computer-executable instructions 106 are performed by the processor 108, performed by multiple processors within the computing device 102, or performed by a processor external to the computing device 102. In some examples, the processor 108 is programmed to execute computer-executable instructions 106 such as those illustrated in the figures described herein, such as FIG. 9. In various examples, the processor 108 is configured to execute one or more of the profile generator 120, cluster generator 122, and recommendation engine 124.

The memory 104 includes any quantity of media associated with or accessible by the computing device 102. In some examples, the memory 104 is internal to the computing device 102. In other examples, the memory 104 is external to the computing device 102 or both internal and external to the computing device 102. For example, the memory 104 can include both a memory component internal to the computing device 102 and a memory component external to the computing device 102. The memory 104 stores data, such as one or more applications 107. The applications 107, when executed by the processor 108, operate to perform various functions on the computing device 102. The applications 107 can communicate with counterpart applications or services, such as web services accessible via the network 134. In an example, the applications 107 represent server-side services of an application executing in a cloud, such as a cloud server.

In some examples, the application 107 is an application for generating an acne profile of a consumer and generating a recommendation for one or more products and/or lifestyle changes to treat the acne. In some examples, the application 107 is an application for generating a hair profile of a consumer and generating a recommendation for one or more products and/or lifestyle changes to care for the hair of the consumer. In some examples, the application 107 is an application for assisting a user experiencing a particular phase of life, such as menopause, and generating a recommendation for one or more products and/or lifestyle changes to address symptoms believed to be caused by the particular phase of life.

In some examples, such as where the application 107 is an application for generating the acne profile and associated recommendation or recommendations, the application 107 further includes image analysis for analyzing an image or images captured by the image capturing device 152. For example, the application 107 may perform specialized image analysis particular to the application 107. Where the application 107 generates an acne profile, the specialized image analysis includes executing a machine learning (ML) or artificial intelligence (AI) based tool to identify and analyze existing acne, blemishes or scars related to historical cases of acne, and so forth. Results from the analyzed image may be used as a variable by the profile generator 120 to generate a profile for the user and place the profile for the user in an existing cluster.

The user interface device 110 includes a graphics card for displaying data to a user and receiving data from the user. The user interface device 110 can also include computer-executable instructions, for example a driver, for operating the graphics card. Further, the user interface device 110 can include a display, for example a touch screen display or natural user interface, and/or computer-executable instructions, for example a driver, for operating the display. The user interface device 110 can also include one or more of the following to provide data to the user or receive data from the user: speakers, a sound card, a camera, a microphone, a vibration motor, one or more accelerometers, a BLUETOOTH® communication module, global positioning system (GPS) hardware, and a photoreceptive light sensor. In a non-limiting example, the user inputs commands or manipulates data by moving the computing device 102 in one or more ways.

The computing device 102 further includes a communications interface device 112. The communications interface device 112 includes a network interface card and/or computer-executable instructions, such as a driver, for operating the network interface card. Communication between the computing device 102 and other devices, such as but not limited to the user device 136, can occur using any protocol or mechanism over any wired or wireless connection.

The computing device 102 further includes a data storage device 114 for storing data, such as, but not limited to one or more profiles 116, clusters 118, and/or interventions 119. The data 114 can be data received from the user device 136 and/or data received, retrieved, or obtained by one or more of the profile generator 120, cluster generator 122, and recommendation engine 124. The profile(s) 116 includes at least one profile for a particular consumer, including data values regarding variables for the consumer. In some examples, the profile, data values, and variables depend on the type of application, or applications, 107 executed on the computing device 102. For example, where the application 107 is a skin health application, the variables may include, but are not limited to, gender, age, a lesion score, a skin tone, whether the consumer has acne marks/scars, a frequency of acne, and a body distribution score. In some examples, one or more scores, such as the lesion score, body distribution score, and so forth, are assigned a color, such as green, red, orange, yellow, etc., rather than a quantified score. Each profile 116 corresponds to a different consumer and is generated by the profile generator 120. The cluster(s) 118 includes at least one cluster that includes a selected set of profiles 116 that have a similarity score above a similarity threshold. Each cluster 118 is generated by the cluster generator 122. The intervention(s) 119 include interventions, as described below, approved for treatment of various health outcomes, such as products, lifestyle adjustments, or a combination of these.

The data storage device 114 can include one or more different types of data storage devices, such as, for example, one or more rotating disks drives, one or more solid state drives (SSDs), and/or any other type of data storage device. The data storage device 114 in some non-limiting examples includes a redundant array of independent disks (RAID) array. In other examples, the data storage device 114 includes a database. The data storage device 114, in this example, is included within the computing device 102, attached to the computing device 102, plugged into the computing device 102, or otherwise associated with the computing device 102. In other examples, the data storage device 114 includes a remote data storage accessed by the computing device 102 via the network 134, such as a remote data storage device, a data storage in a remote data center, or a cloud storage.

The computing device 102 further includes a profile generator 120. In some examples, the profile generator 120 is an example of a specialized processor, or processing unit, implemented on the processor 108. The profile generator 120 generates a profile for a consumer based on data received from the consumer, such as data input on the external device 136 and transmitted to the computing device 102. For example, the profile generator 120 receives raw data from the consumer in various data formats, i.e., textual formats, numerical formats, etc., and converts, or transforms, the received data into a standardized data format. The standardized data is stored in the data storage device 114 as the profile 116.

The computing device 102 further includes a cluster generator 122. In some examples, the cluster generator 122 is an example of a specialized processor, or processing unit, implemented on the processor 108. The cluster generator 122 generates a cluster that includes at least two profiles. To generate a cluster, the cluster generator 122 determines the similarity between a particular profile and each additional profile. In some examples, a profile is added to a particular cluster when the profile has a similarity threshold with the other profiles that are included in the cluster that reaches or exceeds a similarity threshold. The similarity threshold may be a threshold percentage of data values of the profiles that are the same or similar, a threshold number of the data values that are the same, one or more particular values that are the same, or any other suitable threshold for determining similarity. In some examples, the cluster generator 122 generates a persona, or phenotype, associated with each generated cluster. The generated persona includes an artificial profile that represents the data values of the profiles in the cluster.

The computing device 102 further includes a recommendation engine 124. In some examples, the recommendation engine 124 is an example of a specialized processor, or processing unit, implemented on the processor 108. The recommendation engine 124 generates one or more recommendations based on the consumer profile and/or the cluster to which the consumer profile belongs. The recommendation engine 124 generates a recommendation based on the type of system 100 to which the recommendation engine 124 belongs. For example, where the system 100 is a system for generating acne profiles and recommendations, the recommendation engine 124 generates a recommendation for addressing acne. Where the system 100 is a system for generating menopausal profiles and recommendations, the recommendation engine 124 generates a recommendation for addressing menopausal symptoms.

The recommendation engine 124 includes a health outcome identifier 126, an intervention identifier 128, a recommendation generator 130, and a feedback receiver 132. The health outcome identifier 126 identifies a health outcome associated with a particular cluster. In some examples, the health outcomes include a condition, such as acne, dandruff, menopause, and so forth, and a degree, or severity, such as high, mild, widespread, contained, and so forth. In other words, a health outcome may be widespread acne, low dandruff, and so forth. In some examples, the health outcome further includes a frequency, such as often, occasional, etc. In some examples, health outcome further includes additional detail, such as an anticipated underlying cause of the health outcome, such as inflammatory, clogged pores, genetic, environmental, and so forth. In some examples, the identified health outcomes are stored as a value of the cluster 118 in the data storage device 114.

The intervention identifier 128 identifies a particular intervention that, when applied, are anticipated to alleviate or mitigate the identified health outcome based on research data, clinical data, and so forth. Various examples of an intervention include a product, a combination of two or more products, a lifestyle adjustment, a combination of two or more lifestyle adjustments, a combination of at least one product and at least one lifestyle adjustment, and so forth. In some examples, the interventions are stored in the data storage device 114 as interventions 119 crossed reference with health outcomes. In some examples, the interventions 119 are ranked according to an anticipated outcome. For example, an intervention 119 having a greater likelihood of a positive outcome is ranked higher than an intervention 119 having a lower likelihood of a positive outcome. In another example, an intervention 119 having a greater potential effect is ranked higher than an intervention having a lower potential effect.

The recommendation generator 130 generates a recommendation for a consumer associated with a particular profile. In examples where the intervention identifier 128 identifies one potential intervention, the generated recommendation includes the intervention 119 identified by the intervention identifier 128 and instructions for implementation of the identified intervention 119. In examples where multiple potential interventions are identified, the recommendation generator 130 generates a recommendation that includes the highest ranked intervention 119 or a set of the highest ranked interventions. In some examples, the recommendation generator 130 selects an intervention 119 or interventions 119 based on weighing a likelihood of engagement and compliance with the intervention, severity of the health outcome, and immune/holistic health. For example, where a severity of the health outcome is low, the recommendation generator 130 may place a higher weight on engagement and compliance to treat a less severe condition, in contrast to an example where the severity of the health outcome is higher and a greater weight is placed on immune/holistic health that lends toward a stronger intervention that otherwise may have a lower engagement or compliance.

For example, Table 1 illustrates an example of different interventions having scores for different holistic health effects, engagement and compliance (i.e., “adopted”), a mean benefit, and a projected benefit.

TABLE 1

| | Sleep | Gut | Skin | Immune | | Mean | Projected |
|---|---|---|---|---|---|---|---|
| Intervention | Health | Health | Health | Health | Adopted | Benefit | Benefit |
| 1 | 1.04 | 0.99 | 0.90 | 0.92 | 0.97 | 0.96 | 0.96 |
| 2 | 1.01 | 0.98 | 0.97 | 0.98 | 0.72 | 0.99 | 0.99 |
| 3 | 0.98 | 1.01 | 0.97 | 1.05 | 0.45 | 1.00 | 1.00 |
| 4 | 1.03 | 1.05 | 0.98 | 0.99 | 0.35 | 1.01 | 1.00 |
| 5 | 1.09 | 0.90 | 0.99 | 1.00 | 0.91 | 1.00 | 1.00 |
| 6 | 0.98 | 1.00 | 1.00 | 1.01 | 0.13 | 1.00 | 1.00 |
| 7 | 0.99 | 0.97 | 1.01 | 0.98 | 0.56 | 0.99 | 0.99 |
| 8 | 1.00 | 1.05 | 1.03 | 1.03 | 0.88 | 1.03 | 1.02 |
| 9 | 0.92 | 1.04 | 1.04 | 1.01 | 0.65 | 1.00 | 1.00 |
| 10 | 1.07 | 1.10 | 1.05 | 1.04 | 0.78 | 1.07 | 1.05 |
| 11 | 1.04 | 1.03 | 1.05 | 0.97 | 0.31 | 1.02 | 1.01 |
| 12 | 1.02 | 0.97 | 1.10 | 1.09 | 0.90 | 1.05 | 1.04 |
| n | a | b | c | d | x | mean(a, b, | |
| | | | | | | c, d) | |

In some examples, an intervention includes a particular product, such as an acne product, and a lifestyle adjustment. The generated recommendation includes instructions for implementing the intervention, such as a quantity of the acne product to be applied a set number of times per day or per week. For example, the generated recommendation includes the use of two products, a facial cleanser and a moisturizing product, and instructions to use the two products including for the consumer to wash their face, apply a particular amount of the facial cleanser, directly after applying the facial cleanser, applying a particular amount of the moisturizer, waiting a predetermined time, and then applying an additional product, such as a sun protection factor (SPF) product, to avoid acne marks and scarring. It should be understood that this example of a recommendation is presented for illustration only and should not be construed as limiting. Various examples of a generated recommendation are possible without departing from the scope of the present disclosure. In some examples, recommendations for a particular product or products includes a link to a website where the recommended product is available for purchase by the consumer.

The feedback receiver 132 receives feedback regarding a generated recommendation. In some examples, the feedback is received via the user interface device 110. In other examples, the feedback is received via the communications interface device 112 from an external device, such as a user device 136, of the consumer that implements the generated recommendation. The received feedback includes one or more indications regarding the success, failure, and/or viability of the generated recommendation. In the example above where the generated recommendation includes the facial cleanser and moisturizing product and associated instructions, the feedback may be that the generated recommendation resulted in positive results, e.g., reduced acne, negative results, e.g., no change in acne or increased acne, or non-viability, such as the generated recommendation was difficult to implement due to complex instructions, too much time was required to correctly follow the instructions, and so forth. In some examples, feedback is received through the completion of a user survey, where a consumer provides scaled ratings for various elements of the recommendation, such as a scale of one to ten, one to five, and so forth. In other examples, feedback is received based on user data that maps whether the recommended product was purchased through the provided link, whether the recommended product was purchased at a later time, whether the recommended product was purchased multiple times, and so forth. In these examples, the feedback receiver 132 analyzes the received feedback and provides the analyzed feedback to the recommendation generator 130.

Based on the received feedback, the recommendation generator 130 is updated based on the received feedback. For example, positive feedback is used to reinforce the generated recommendation, while negative feedback or feedback that a recommendation is non-viable is implemented so that a future recommendation for the consumer and/or other profiles in the same cluster as the consumer includes a different product, different combination of products, different instructions for implementing the product or products, and so forth.

In some examples, the received feedback is further used by the profile generator 120 to update the profile. For example, feedback received that indicates a lower level of acne automatically updates the profile for the consumer to reflect the lowered level of acne. In some examples, based on the updated profile, the cluster generator 122 re-clusters the updated profile based on the updated information. For example, the cluster generator 122 re-clusters the updated profile into a new cluster that includes other profiles with similar acne levels. Based on the updated cluster for the profile, the recommendation generator 130 generates an updated recommendation for the consumer to reflect the updated profile and cluster, including but not limited to a product, a combination of two or more products, a lifestyle adjustment, a combination of two or more lifestyle adjustments, a combination of at least one product and at least one lifestyle adjustment, and so forth, where the products and lifestyle adjustments may be the same or different than the originally recommendation products or lifestyle adjustments, respectively.

The user device 136 is another example of a computing device, separate from and external of the computing device 102. In some examples, the user device 136 includes a mobile computing device or any other portable device. A mobile computing device includes, for example but without limitation, a mobile telephone, laptop, tablet, computing pad, netbook, gaming device, and/or portable media player. The user device 136 can also include less-portable devices such as servers, desktop personal computers, kiosks, or tabletop devices. Additionally, the user device 136 can represent a group of processing units or other computing devices.

In some examples, the user device 136 includes at least one processor 142, a memory 138 that includes the computer-executable instructions 140, and a user interface device 144. The processor 142 includes any quantity of processing units and is programmed to execute the computer-executable instructions 140. The computer-executable instructions 140 are performed by the processor 142, performed by multiple processors 142 within the user device 136, or performed by a processor 142 external to the user device 136. In some examples, the processor 142 is programmed to execute computer-executable instructions 140 such as those illustrated in the figures described herein, such as FIG. 9. In various examples, the processor 142 is configured to execute an application 150, which is a client-side version of the application 107.

The memory 138 includes any quantity of media associated with or accessible by the user device 136. In some examples, the memory 138 is internal to the user device 136. In other examples, the memory 138 is external to the user device 136 or both internal and external to the user device 136. For example, the memory 138 can include both a memory component internal to the user device 136 and a memory component external to the user device 136. The memory 138 stores data, such as one or more applications 150. The applications 150, when executed by the processor 142, operate to perform various functions on the user device 136. The applications can communicate with counterpart applications or services, such as web services accessible via the network 134. In an example, the applications represent downloaded client-side applications that correspond to server-side services executing in a cloud, such as a cloud server.

In some examples, the application 150 may be an example of the application 107. In other words, where the application 150 is an application for generating the acne profile and associated recommendation or recommendations, the application 150 further includes image analysis for analyzing an image or images captured by the image capturing device 152. For example, the application 150 may perform specialized image analysis particular to the application 150. Where the application 150 generates an acne profile, the specialized image analysis includes executing a ML or AI based tool to identify and analyze existing acne, blemishes or scars related to historical cases of acne, and so forth. Results from the analyzed image may be used as a variable by the profile generator 120 to generate a profile for the user and place the profile for the user in an existing cluster.

The user interface device 144 includes a graphics card for displaying data to a user and receiving data from the user. The user interface device 144 can also include computer-executable instructions, for example a driver, for operating the graphics card. Further, the user interface device 144 can include a display, for example a touch screen display or natural user interface, and/or computer-executable instructions, for example a driver, for operating the display. The user interface device 144 can also include one or more of the following to provide data to the user or receive data from the user: speakers, a sound card, a camera, a microphone, a vibration motor, one or more accelerometers, a BLUETOOTH® communication module, global positioning system (GPS) hardware, and a photoreceptive light sensor. In a non-limiting example, the user inputs commands or manipulates data by moving the user device 136 in one or more ways.

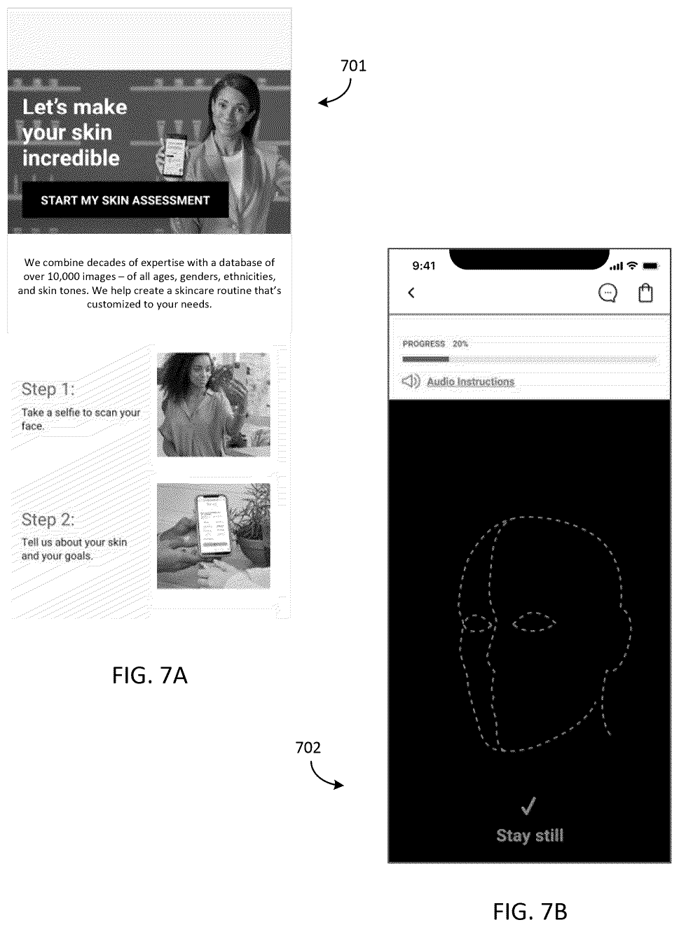

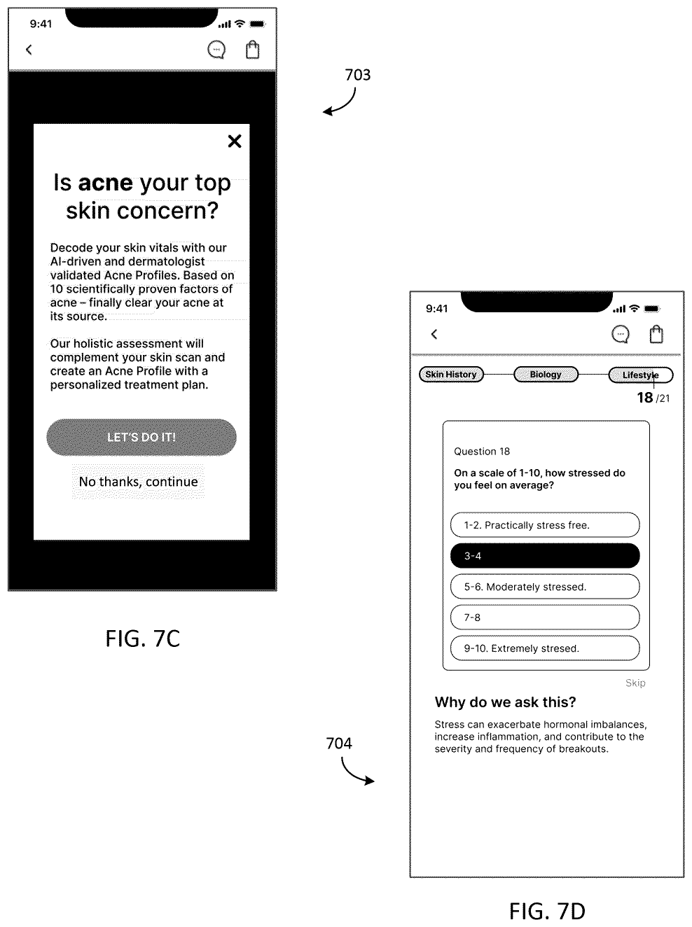

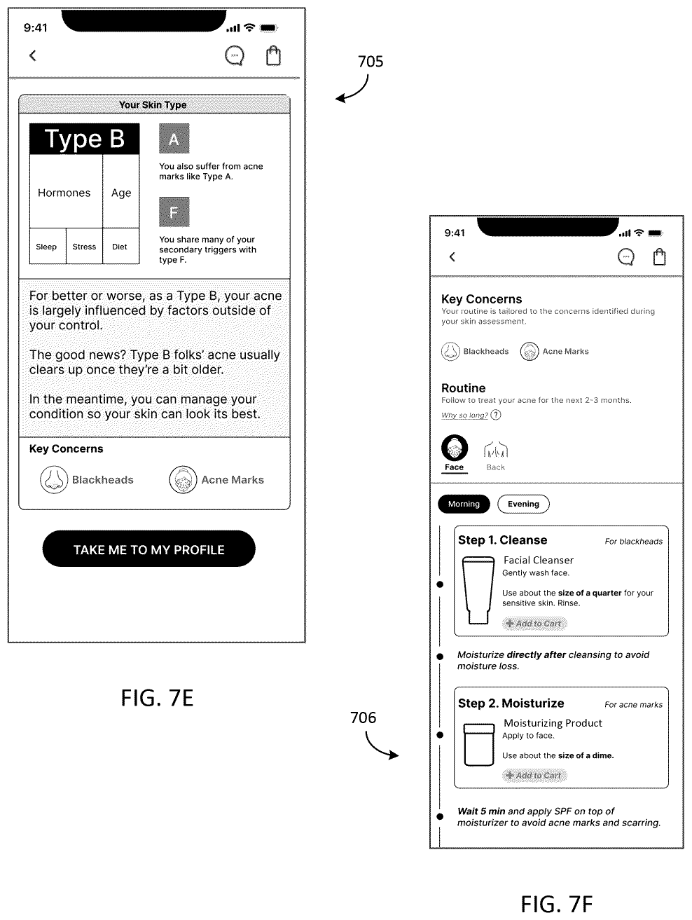

In some examples, the user interface device 144 is configured to launch and display a visualization of the application 150, such as illustrated in FIGS. 7A-7F. For example, the processor 142 can execute the computer-executable instructions 140 stored in the memory 138 to execute the application 150 and the visualization of the application 150, illustrated in FIGS. 7A-7F, is presented via the user interface device 144.

The user device 136 further includes a communications interface device 146. The communications interface device 146 includes a network interface card and/or computer-executable instructions, such as a driver, for operating the network interface card. Communication between the user device 136 and other devices, such as but not limited to the computing device 102, can occur using any protocol or mechanism over any wired or wireless connection.

The user device 136 further includes a data storage device 148 for storing data, such as, but not limited to user provided data associated with a profile 116 of a consumer who is the user of the user device 136. The data can be data received via the user interface device 144 and/or data received, retrieved, or obtained from the computing device 102.

The data storage device 148 can include one or more different types of data storage devices, such as, for example, one or more rotating disks drives, one or more solid state drives (SSDs), and/or any other type of data storage device. The data storage device 148 in some non-limiting examples includes a redundant array of independent disks (RAID) array. In other examples, the data storage device 148 includes a database. The data storage device 148, in this example, is included within the user device 136, attached to the user device 136, plugged into the user device 136, or otherwise associated with the user device 136. In other examples, the data storage device 148 includes a remote data storage accessed by the user device 136 via the network 134, such as a remote data storage device, a data storage in a remote data center, or a cloud storage.

The user device 136 further includes an image capturing device 152. In some examples, the image capturing device 152 is a camera operable to capture still images and/or video of a consumer's skin. In some examples, the captured images and/or video is used by the recommendation engine 124 to enhance the generated recommendation, as described herein. For example, the captured images and/or video is included in the profile 116 of the user stored in the data storage device 114 and used to provide objective detail regarding the consumer's skin, in comparison to subjective detail that is provided by the user, for example in response to a questionnaire.

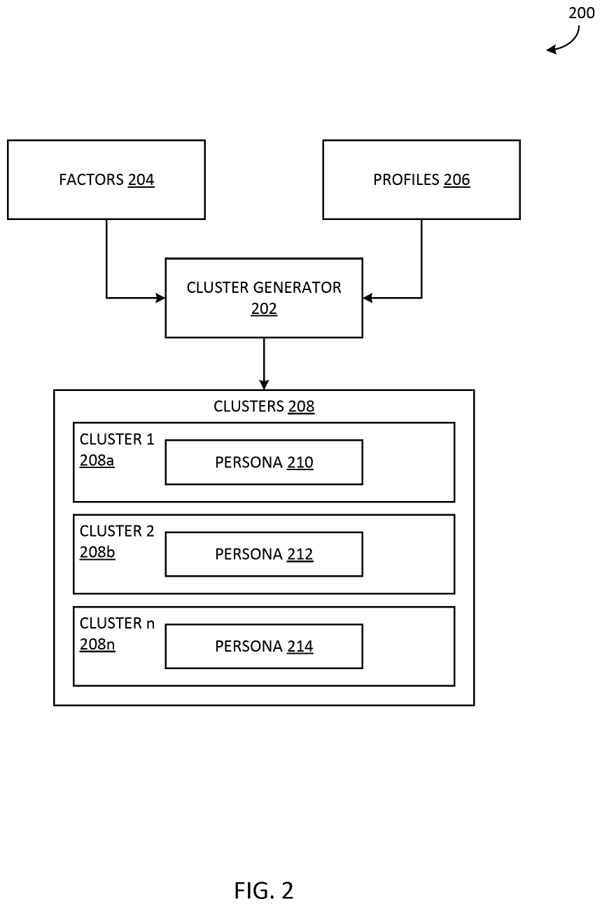

FIG. 2 illustrates an example system for generating a plurality of personas. The example system illustrated in FIG. 2 is for illustration only and should not be construed as limiting. Various examples of the system 200 may be implemented without departing from the scope of the present disclosure. In various examples, the system 200 is implemented by one or more elements of the system 100, such as the cluster generator 122.

The system 200 includes a cluster generator 202. In some examples, the cluster generator 202 is an example of the cluster generator 122 illustrated in FIG. 1. The cluster generator 202 receives, as inputs, one or more factors 204 and one or more profiles 206 and generates one or more clusters 208 based on the combination of factors 204 and profiles 206. The factors, or variables, 204 include different factors for a particular condition, such as acne, that may contribute to a likelihood of developing the condition, exacerbating the condition, and so forth. In examples where the condition is acne, factors gender, age, a lesion score that is calculated based on quantities of non-inflammatory lesions, inflammatory lesions, and clogged pores, a skin tone, whether the consumer has acne marks/scars, a frequency of acne, and a body distribution score that is calculated based on the total number of acne locations on the face and body of a consumer. In some examples, additional factors 204 may include health history of the consumer, including mental health, sun exposure history, and other environmental factors. Each profile 206 includes data related to each factor 204 for the particular consumer associated with the profile 206. For example, each profile 206 includes consumer data related to the gender of the consumer, age of the consumer, lesion score of the consumer, skin tone of the consumer, acne marks/scars of the consumer, frequency of acne of the consumer, and body distribution score of the consumer.

The cluster generator 202 implements a clustering algorithm, or set of algorithms, to determine the similarity between a particular profile and each additional profile. In some examples, a profile is added to a particular cluster, or a new cluster is generated, when the profile has a similarity threshold with the other profiles that are included in the cluster that reaches or exceeds a similarity threshold. The similarity threshold may be a threshold percentage of data values of the profiles that are the same or similar, a threshold number of the data values that are the same, one or more particular values that are the same, or any other suitable threshold for determining similarity.

In examples where the cluster generator 202 initially generates clusters, the cluster generator 202 identifies one or more identifying features of a persona of the cluster. The persona is an artificial profile that represents the data values of the profiles in the cluster. For example, a persona for a first cluster includes males ages 28-32 with medium lesion scores. The cluster generator 202 identifies profiles that match aspects of the persona and then separates those profiles into one or more clusters based on additional factors. In this example, a first cluster 208a is generated for profiles of males ages 28-32 with medium lesion scores and an acne frequency of “often”, a second cluster 208b is generated for profiles of males ages 28-32 with medium lesion scores and an acne frequency of “sometimes”, and a third cluster 208n is generated for profiles of males ages 28-32 with medium lesion scores and an acne frequency of “rare”. A first persona 210 is associated with the first cluster 208a, a second persona 212 is associated with the second cluster 208b, and a third persona 214 is associated with the third cluster 208n. It should be understood that the present example is presented for illustration only. In various examples, various clusters are generated based on a variety of different factors without departing from the scope of the present disclosure. In particular, eight example clusters are presented in Table 2 below.

TABLE 2

| | Cluster | Cluster | Cluster | Cluster | Cluster | Cluster | Cluster | Cluster |
|---|---|---|---|---|---|---|---|---|
| | 1 | 2 | 3 | 4 | 5 | 6 | 7 | 8 |
| Gender | M | F | F | F | M | F | BOTH | BOTH |
| Avg | 31 | 25 | 29 | 25 | 27 | 27 | 29 | 24 |
| Age | | | | | | | | |
| Lesion | Medium | Medium | Medium | Medium | Medium | Medium | Medium | Medium |
| Score | | | | | | | | |
| Skin | Light | Medium | Light | Medium | Medium | Light | Light | Dark |
| Tone | | | | | | | | |
| Acne | Yes | Yes | Yes | Yes | Yes | Yes | No | Yes |
| Marks/ | | | | | | | | |
| Scars | | | | | | | | |
| Acne | Often | Sometimes | Sometimes | Often | Often | Often | Sometimes | Often |
| Frequency | | | | | | | | |
| Body | Low | Medium | Low | Medium | Medium | Medium | Low | Medium |
| Dist. | | | | | | | | |
| Score | | | | | | | | |

Accordingly, the architectural structure of the clusters 208 and personas 210-214 provide an improved set of indexes that are stored in the data storage device 114 that facilitate improved retrieval by one or more elements of the recommendation engine 124. Improving the retrieval of data associated with one or more clusters 208 and/or one or more personas 210-214 enables more effective association of a new profile with an existing cluster as well as more effective re-association of an existing profile with a different cluster based on new data being received. The retrieval of the data is improved by storing the clusters 208, i.e., the clusters 118, in the data storage device 114 with the associated personas 210-214 embedded with the clusters 208, which reduces the consumption of computing resources required to associate an updated profile with a new cluster. Clusters 208 may be stored with in the data storage device 114 with tags indicating similarities between different clusters 208 so that the cluster generator 202 may identify a new cluster, or set of new clusters, with a high probability for an updated profile to be associated with. This in turn enables the cluster generator 122 to learn, over time, that particular changes to profiles in a particular cluster 208 historically lead to the profile being associated with a particular new cluster. This results in a reduced consumption of computing resources for associating an existing profile with a new cluster based on new data being received.

In examples where the cluster generator 202 receives a new or updated profile to include in an existing cluster, the cluster generator 202 executes a clustering algorithm to compare the received profile to other profiles in different clusters. In some examples, the profile is added to the cluster with which the received profile has the highest similarity score. In other examples, the highest similarity score is compared to a similarity threshold to determine whether the profile is similar enough to the profiles of the closest cluster to be included in the cluster. Where the similarity score is equal to or exceeds the similarity threshold, the profile is added to the cluster. Where the similarity score is less than the similarity threshold, the profile is not similar enough to the profiles in the cluster to be included and the cluster generator 202 generates a new cluster for the profile.

In some examples, the cluster generator 202 implements a two-stage process to strategically prune a feature space for biological features, given that data may include a high variety of features, some of which are highly correlated with a particular condition, such as acne, and others which are less associated with the particular condition. The two-stage process includes i) a hierarchical feature collapse, and ii) a selection of top features by performing a principal component analysis (PCA). In the hierarchical feature collapse, the cluster generator 202 groups features together are more similar to one another, reflecting the strength of similarity between the features on a vertical axis. The number of resulting feature clusters is determined based on where the threshold on the vertical axis is cut. Features with a maximum variance are taken in the scenario where there are multiple elements in the feature cluster. In some examples, a more aggressive threshold is taken to further collapse the feature space. In other examples, a more conservative threshold is taken so as to only eliminate highly redundant features.

PCA is an unsupervised machine learning method to partition the variability in the data on non-overlapping axes consisting of linear combinations of the features such that the first few axes account for the most variability. The data is normalized by centering and scaling the data, which improves how informative the PCA. Otherwise, some features on a larger scale may dominate the axes. In some examples, a first number of features, such as the first then features in the first three principal components, are selected to identify the top features.

In some examples, the clusters generated by the cluster generator 202 are generated and output as a visualization, such as a heatmap. For visualization of the clusters, the biological dataset is scaled by a maximum feature importance for each of the features across the first three principal components. This effectively weights the raw data by the amount of variability the feature accounts for in the biological dataset and thus provides a better visual representation. In some examples, the clusters are visualized as clouds of like individuals as opposed to discrete clusters, given the continuous nature of the biological data, such as the number of lesions, age, outbreak frequency, and so forth. In some examples, an additional model is used to further cluster the data. For example, a partition around medoids (PAM) may be selected, which is robust to outliers, stable, fast, and capable of handling different data types. Various performance metrics, such as the Silhouette index, Calinski-Harabasz, or any other suitable clustering performance metrics, may be used to determine cluster size. As the biology underlying acne is continuous, literal interpretation of these discrete clustering metrics is inappropriate. Instead, using them as guidelines for a range of k may provide more informative clustering and analysis. The choice of k is determined based on a balance between visually inspecting the plots of these metrics over k and marketing bandwidth for acne personalization. The generated heatmap may then display the features by cluster, where color intensity corresponds to more extreme values, a first color corresponds to higher values, and a second color corresponds to lower values.

In summary, the cluster generator 202 performs the clustering by performing a hierarchical feature collapse to remove redundant features, taking top PCA features as selected features, scaling the PCA visualization by weights of top features, applying an additional model, such as PAM, to cluster the scaled data of selected features, choosing a value k by using various performance metrics such as the Silhouette index, Calinski-Harabasz, or other marketing resource constraints, and re-clustering with an optimal k value and using the cluster labels for analysis.

In some examples, certain biological features, also referred to herein as inclusion features, are included in the feature space utilized by the cluster generator 202 due to healthcare providers or practitioners considering them particularly pertinent to the particular condition or removed from the feature space utilized by the cluster generator 202 due to healthcare providers or practitioners considering them not particularly relevant to the particular condition. In some examples, the biological feature model includes upweighted features to emphasize a particular feature or features. In some examples, the biological feature model further includes skin conditions, such as atopic dermatitis, psoriasis, and so forth, in addition to the inclusion features described herein.

In some examples, the cluster generator 202 further clusters behavior determined to relate specifically to the particular condition in question, such as acne. To do so, the cluster generator 202 i) correlates biological and behavior features, ii) for each mapped biological feature, identifies behavioral features that map to the biological feature above a set threshold, iii) uses the highest mapped behaviors for a cluster, and iv) applies the clustering process described herein for the various factors except for the behavioral feature collapse, as feature filtering has already been performed. For example, the cluster generator 202 correlates the biological and behavioral features and identifies which behavioral features have the highest correlations to biological features, which then serve as reasonable proxies for underlying acne biology. The heatmap of the correlation between biological and behavioral features shows that the behavioral features are meaningfully clustered by the biology. In other words, there are clear groupings of behavioral questions/themes, which provides positive feedback of the clustering approach. Although in some examples the absolute correlation between the biological and behavioral features may be relatively low, this signal is meaningful as real-world correlations, particularly with humans, tend to be much weaker and harder to measure than lab controlled experiments.

In some examples, the behavioral model is combined with the biological model to develop a single model that models the dependence of the biological and behavioral features. In this example, a lighter hierarchical feature collapse is performed, as the feature space dramatically expands with the feature union. Using this reduced feature set, the same set of clustering procedures are performed as discussed herein, with the exception of the PCA feature selection stage, which is performed separately in the individual models.

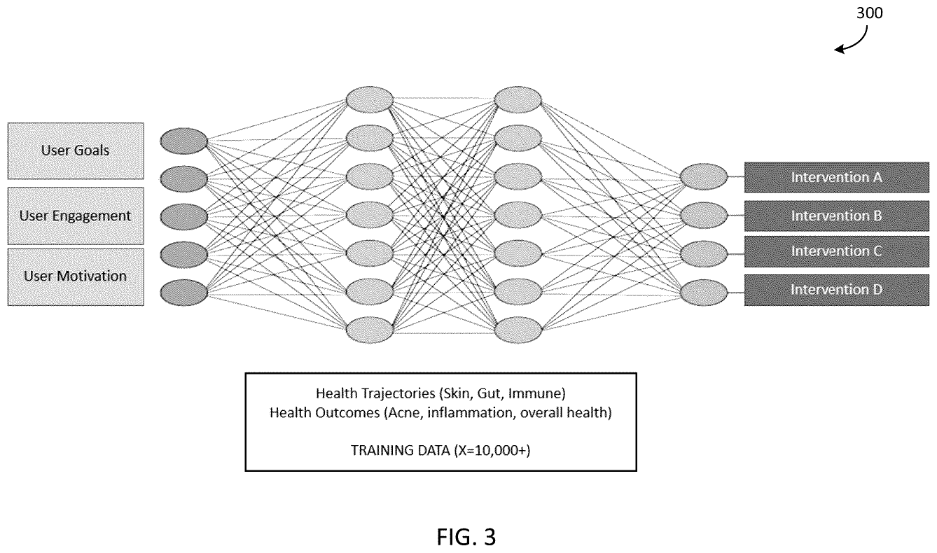

FIG. 3 illustrates an example of a recommendation engine for generating interventions for a particular profile. The example recommendation engine 124 illustrated in FIG. 3 is for illustration only and should not be construed as limiting. Various examples of the recommendation engine 124 may be implemented without departing from the scope of the present disclosure.

As described herein, the recommendation engine 124 generates one or more recommendations based on the consumer profile and/or the cluster to which the consumer profile belongs. FIG. 3 illustrates examples of inputs received by the recommendation engine 124 in addition to the factors 204 for a particular profile 206 that are used to generate one or more recommended interventions for a particular profile. For example, various examples of inputs the recommendation engine 124 receives and uses include, but are not limited to, user goals, user engagement, and user motivation. User goals are examples of what a user wants to accomplish. In examples where the system 100 is an acne solution, example user goals include, but are not limited to, reducing acne frequency, reducing or removing existing acne scars, reducing a lesion score, reducing a body distribution score, and preventing new acne from appearing. User engagement refers to a user's historical and/or anticipated engagement with the system 100, such as a likelihood that a consumer will implement generated recommendations, provide feedback regarding implemented recommendations, and so forth. User motivation refers to a reasoning why the consumer initially engaged and/or continues to engage the system 100. User motivation arises from a psychological profile of an individual. For example, a target goal may be provided but depending on the user motivation, this goal may or may not be expected to be achieved. In one example, a goal is medication adherence, which is traditionally difficult to achieve due to product routines and regimens. Where a user's underlying motivations are understood, different products may be recommended based on the ability of a user, or anticipated ability of a user, to adhere to.

Based on the received inputs, the recommendation engine 124 generates a recommendation that includes at least one intervention, such as intervention A, intervention B, intervention C, intervention D, and so forth that, when implemented, addresses an anticipated health outcome for the consumer. As described herein, an intervention include a product, a combination of two or more products, a lifestyle adjustment, a combination of two or more lifestyle adjustments, a combination of at least one product and at least one lifestyle adjustment, and so forth.

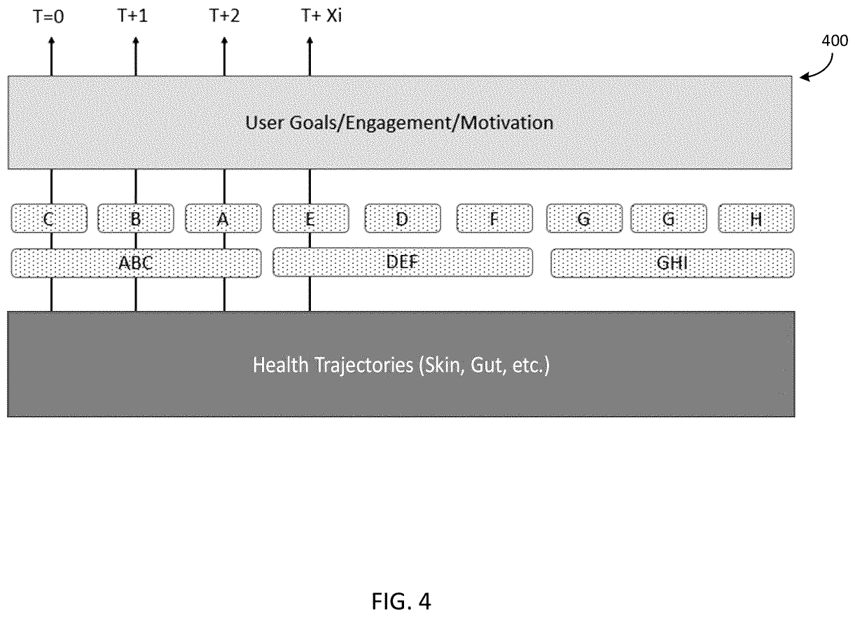

FIG. 4 illustrates an example timeline of a profile updating over time. The example timeline 400 illustrated in FIG. 4 is provided for illustration only and should not be construed as limiting. Various examples of the timeline 400 may be implemented without departing from the scope of the present disclosure.

The timeline 400 illustrates a combination of health trajectories, user goals, engagement, and motivation, rankings, and models over time. As time progresses, a consumer's goals, engagement, and motivation, rankings may change. In addition, the cluster in which the profile of the consumer is placed may change based on the consumer's changing age, updates to the consumer's profile such as an acne frequency or lesion score being reduced over time due to the implementation of a recommended intervention or interventions, and so forth.

As shown in FIG. 4, at an initial time, T=0, the consumer has a ranking of C and a particular recommendation model ABC is implemented to generate a recommendation. At a second time, T+1, as the consumer goals change, the consumer has an updated ranking of B, but the recommendation model ABC is still implemented to generate the recommendation. Additional changes are shown at additional times T=2 and T=Xi.

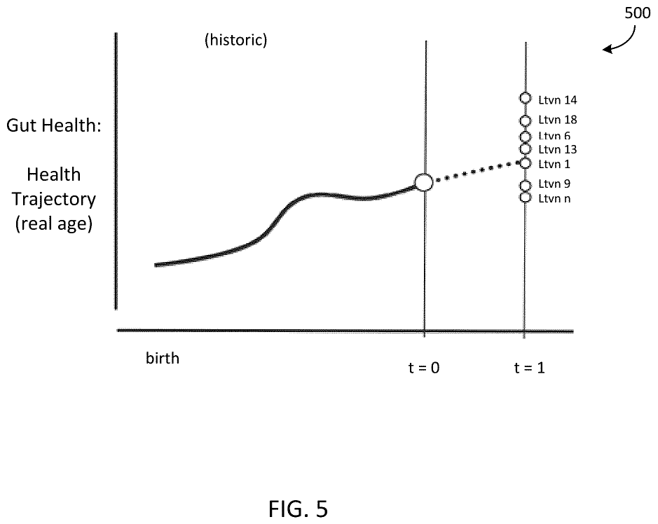

FIG. 5 illustrates an example health outcome over time. The example health outcomes illustrated in FIG. 5 illustrates a projected healthcare outcome, for example gut health, of potential interventions if deployed at an initial time, such as T=0, as shown in FIG. 4. In some examples, recommendation engine 124 utilizes the projected healthcare outcomes to identify a recommendation that is projected to have a greatest effect if deployed, measured by the slope of the projected curve relative to other potential interventions.

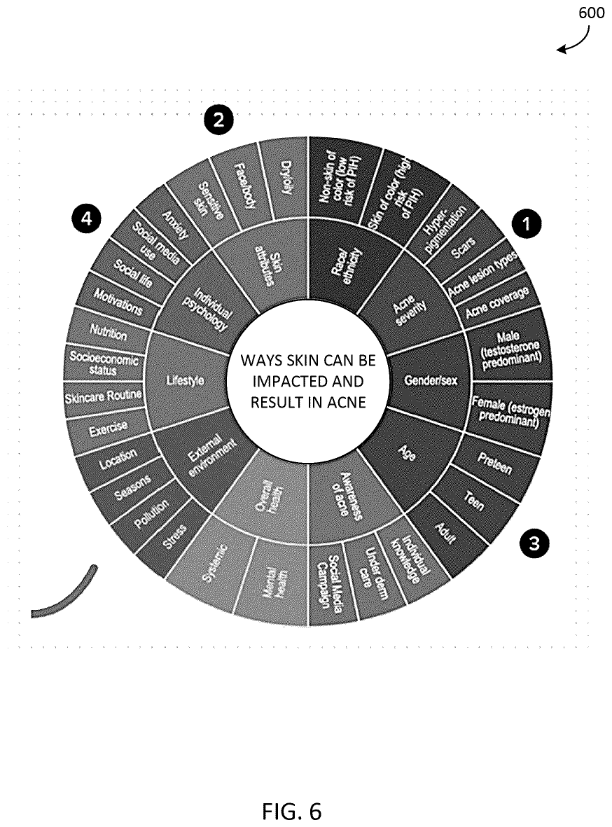

FIG. 6 illustrates an example for building profiles and associated clusters for one or more example profiles. The example illustrated in FIG. 6 is provided for illustration only and should not be construed as limiting. Various examples may be used without departing from the scope of the present disclosure.

In particular, FIG. 6 illustrates a wheel that represents different factors, or variables, and associated sub-factors that contribute to each factor. For example, one factor is identified as external environment. Sub-factors that contribute to the external environment include location, seasons, pollution, diet, and stress. In another example, a factor is identified as skin attributes, and sub-factors that contribute to the skin attributes include dry or oily, face or body, and whether the consumer has sensitive skin. In another example, a factor is identified as the consumer's lifestyle, and sub-factors that contribute to the lifestyle include nutrition, socioeconomic status, skincare routine, and exercise habits. The profile generator 120 generates a descriptive profile including details of the consumer using the factors and the sub-factors, the cluster generator 122 generates clusters based on similar profiles and identified relationships between the factors and sub-factors, and the recommendation engine 124 generates recommendations as described herein that account for the various factors and sub-factors.

FIGS. 7A-7F illustrate example user interfaces (UIs) of a flow including creating a profile, performing a facial scan, receiving data, and generating a recommendation. The example UIs illustrated in FIGS. 7A-7F are provided for illustration only and should not be construed as limiting. Various examples may be used without departing from the scope of the present disclosure. In some examples, the example UIs illustrated in FIGS. 7A-7F are examples of the user interface device 144 illustrated in FIG. 1 presenting a client-side application 150.

FIG. 7A illustrates a first UI 701. The first UI 701 is an example of a landing page of the application 150 that introduces the application 150 and provides a consumer with information regarding the application 150. For example, the first UI 701 includes background information, including that the application 150 combines expertise with a database of images to create a customized skincare routine. The first UI 701 further includes information regarding a first step, at which the user device 136 is used to capture an image of the consumer's face, and a second step at which the consumer provides information regarding their skin and goals.

FIG. 7B illustrates a second UI 702. The second UI 702 is an example of the first step identified in the first UI 701, where the image capturing device 152 is used to capture an image of the consumer. The second UI 702 includes instructions for performing the scan, a progress bar indicating the progress of the facial scan, and a selectable icon that, upon selection, provides audio instructions for executing the facial scan.

FIG. 7C illustrates a third UI 703. The third UI 703 provides the consumer with information regarding completing a holistic assessment and generating a profile 116, and selectable icons for both beginning the holistic assessment and to continue without completing the holistic assessment that, upon selection, initiate the holistic assessment and continue without initiating the holistic assessment, respectively. FIG. 7D illustrates a fourth UI 704 that includes an aspect of a questionnaire. In some examples, the questionnaire includes a set of diagnostic questions. In particular, the fourth UI 704 illustrates an example question of the questionnaire regarding a consumer's average stress levels, with multiple selectable options representing different levels of average stress. Although illustrated in FIG. 7D as using multiple options, various examples are possible. Some examples may include binary selections, such as yes/no or true/false, range selections, such as one to five or one to ten, or open-ended questions where a consumer provides an answer in their own words by typing into a text box. The profile generator 120 receives the answers provided to the questions of the questionnaire and generates the profile 116 based on the received answers and captured facial images.

FIG. 7E illustrates a fifth UI 705. The fifth UI 705 presents results of the questionnaire that are implemented into the particular profile 116 for the consumer. For example, the fifth UI 705 presents a skin type for the consumer, including one or more factors that contribute to acne for the particular skin type, as well as aspects of additional skin types with which the consumer shares similar characteristics. The fifth UI 705 further includes general information regarding the presented skin type, such as causes of acne for the skin type, a prognosis for resolving symptoms, options for managing the acne, and key concerns for the particular skin type.

FIG. 7F illustrates a sixth UI 706. The sixth UI 706 includes a generated recommendation for the consumer that includes a skincare routine and a time frame for which the recommended routine is recommended to be followed. The skincare routine includes one or more recommended products along with detailed instructions regarding application instructions for the recommended products and an order for which the recommended products are to be used.

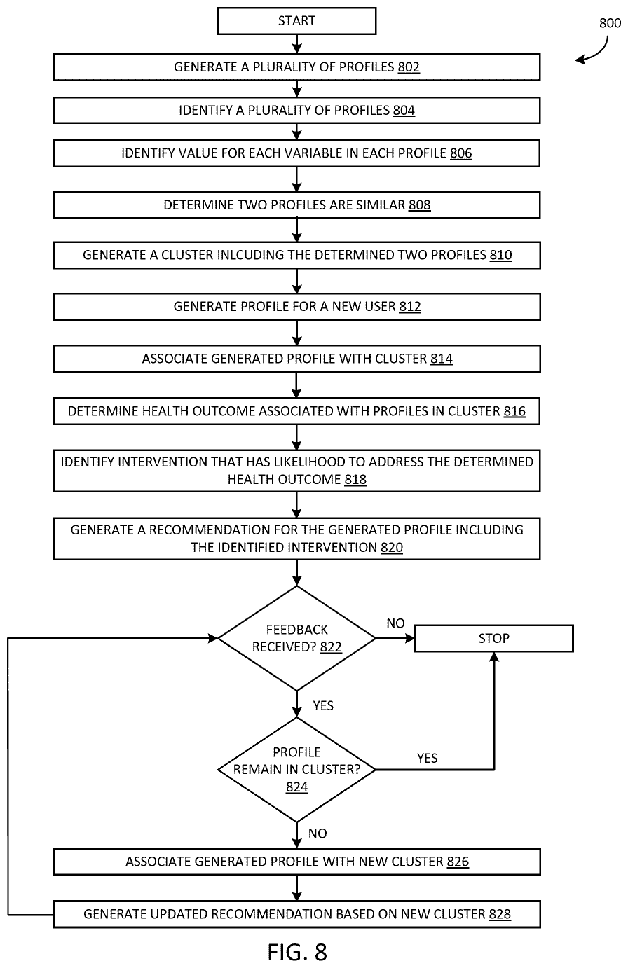

FIG. 8 illustrates an example computer-implemented method of generating one or more recommendations for a profile. The computer-implemented method 800 is presented for illustration only and should not be construed as limiting. Other examples of the computer-implemented method 800 can be used without departing from the scope of the present disclosure. The computer-implemented method 300 can be implemented by one or more electronic devices described herein, such as the computing device 102.

The computer-implemented method 800 begins by the profile generator 120 generating a plurality of profiles 116 in operation 802. As described herein, the profile generator 120 generates a separate profile 116 for each respective consumer based on data received from the consumer, such as data input on the external device 136 and transmitted to the computing device 102. Each newly generated profile 116 is stored in the data storage device 114.

In operation 804, the cluster generator 122 identifies a plurality of generated profiles 116. In some examples, the cluster generator 122 identifies the plurality of generated profiles 116 automatically as the profile is generated. In other examples, the cluster generator 122 identifies the plurality of generated profiles 116 in response to a trigger event, such as a time of day occurring. For example, the cluster generator 122 identifies the plurality of generated profiles 116 at a regular interval, such as the same time each day, once a week, once every two weeks, and so forth.

In operation 806, the cluster generator 122 identifies a value for each variable in each profile 116. As described herein, variables for the consumer include, but are not limited to, gender, age, a lesion score, a skin tone, whether the consumer has acne marks/scars, a frequency of acne, and a body distribution score. A value is a value of a particular variable. For example, a value for the variable of gender includes male, female, or not specified. A value for the variable of age includes the numerical age of the consumer.

In operation 808, the cluster generator 122 determines that two profiles 116 are similar. In some examples, the cluster generator 202 implements a clustering algorithm to determine the similarity between a particular profile and each additional profile. For example, the cluster generator 202 identifies one or more identifying features of a persona of the cluster. The cluster generator 202 then identifies profiles that match aspects of the persona and then separates those profiles into one or more clusters based on additional factors. In operation 810, generates the cluster 118 including the determined two profiles 116. The generated cluster 118 is stored in the data storage device 114.

In operation 812, the profile generator 120 generates a new profile for a new user. For example, as illustrated in FIGS. 7A-7F, the profile generator 120 generates a new profile for a consumer based on one or more of a facial scan performed by the image capturing device 152 and information received from the consumer indicating their health history, demographic information, skin care history, environmental factors, and so forth. The newly generated profile 116 is stored in the data storage device 114.

In operation 814, the cluster generator 122 associates the newly generated profile 116 with an existing cluster 118. For example, the cluster generator 202 executes a clustering algorithm to compare the received profile to other profiles in different clusters. In some examples, the profile is added to the cluster with which the received profile has the highest similarity score. In other examples, the highest similarity score is compared to a similarity threshold to determine whether the profile is similar enough to the profiles of the closest cluster to be included in the cluster. Where the similarity score is equal to or exceeds the similarity threshold, the profile is added to the cluster. Where the similarity score is less than the similarity threshold, the profile is not similar enough to the profiles in the cluster to be included and the cluster generator 202 generates a new cluster for the profile. The cluster 118 is updated in the data storage device 114 to include the newly generated profile 116.

In operation 816, the health outcome identifier 126 identifies a health outcome associated with the cluster 118 in which the newly generated profile 116 was included. In some examples, the health outcomes include a condition, such as acne, dandruff, and so forth, and a degree, or severity, such as high, mild, widespread, contained, and so forth. In other words, a health outcome may be widespread acne, low dandruff, and so forth. In some examples, the health outcome further includes a frequency, such as often, occasional, etc. In some examples, health outcome further includes additional detail, such as an anticipated underlying cause of the health outcome, such as inflammatory, clogged pores, genetic, environmental, and so forth. In some examples, the identified health outcomes are stored as a value of the cluster 118 in the data storage device 114.

In operation 818, the intervention identifier 128 identifies an intervention that has a likelihood to address the determined health outcome. For example, the intervention identifier 128 identifies a particular intervention that, when applied, are anticipated to alleviate or mitigate the identified health outcome based on research data, clinical data, and so forth. Various examples of an intervention include a product, a combination of two or more products, a lifestyle adjustment, a combination of two or more lifestyle adjustments, a combination of at least one product and at least one lifestyle adjustment, and so forth. In some examples, the interventions are stored in the data storage device 114 as interventions 119 crossed reference with health outcomes.

In operation 820, the recommendation generator 130 generates a recommendation for the profile 116, including the identified intervention 119. The generated recommendation includes the intervention 119 identified by the intervention identifier 128 and instructions for implementation of the identified intervention 119. In some examples, an intervention includes a particular product, such as an acne product, and a lifestyle adjustment. The generated recommendation includes instructions for implementing the intervention, such as a quantity of the acne product to be applied a set number of times per day or per week. For example, the generated recommendation includes the use of two products, a facial cleanser and a moisturizing product, and instructions to use the two products including for the consumer to wash their face, apply a particular amount of the facial cleanser, directly after applying the facial cleanser, applying a particular amount of the moisturizer, waiting a predetermined time, and then applying an additional product, such as a sun protection factor (SPF) product, to avoid acne marks and scarring. In some examples, the generated feedback is transmitted to the user device 136 and presented to the consumer, for example as illustrated in FIG. 7F.

In operation 822, the feedback receiver 132 determines whether feedback has been received regarding the generated and presented recommendation. Received feedback may include one or more indications regarding the success, failure, and/or viability of the generated recommendation. In an example where the generated recommendation includes the facial cleanser and moisturizing product and associated instructions, as illustrated in FIG. 7F, feedback may be that the generated recommendation resulted in positive results, e.g., reduced acne, negative results, e.g., no change in acne or increased acne, or non-viability, such as the generated recommendation was difficult to implement due to complex instructions, too much time was required to correctly follow the instructions, and so forth. In some examples, feedback is received through the completion of a user survey, where a consumer provides scaled ratings for various elements of the recommendation, such as a scale of one to ten, one to five, and so forth. In other examples, feedback is received based on user data that maps whether the recommended product was purchased through the provided link, whether the recommended product was purchased at a later time, whether the recommended product was purchased multiple times, and so forth. In examples where feedback is not received, the computer-implemented method 800 terminates.

In examples where feedback is received, in operation 824 the cluster generator 122 analyzes the received feedback and determines whether changes to the profile 116 result in the profile 116 remaining in the cluster 118. For example, the cluster generator 122 repeats the process described in operation 814 and then determines whether the profile 116 is still most similar to the initial cluster 118 or a new cluster 118. In examples where the changes to the profile 116 do not result in a significant enough change for the profile to move to a different cluster, i.e., the profile 116 remains in the same cluster 118, the computer-implemented method 800 terminates. In examples where the changes to the profile 116 do result in a significant enough change for the profile to move to a different cluster, the computer-implemented method 800 proceeds to operation 826 and the cluster generator 122 associates the profile with a new cluster 118. Then, in operation 828, the recommendation generator 130 generates an updated recommendation for the profile 116 based on the profile 116 being associated with the new cluster 118. The updated recommendation is presented to the consumer, such as via the user device 136, and the computer-implemented method 800 returns to operation 822 to determine whether feedback is received regarding the updated recommendation.

### Example Operating Environment

FIG. 9 is a block diagram of an example computing device 900 for implementing aspects disclosed herein and is designated generally as computing device 900. Computing device 900 is an example of a suitable computing environment and is not intended to suggest any limitation as to the scope of use or functionality of the examples disclosed herein. Neither should computing device 900 be interpreted as having any dependency or requirement relating to any one or combination of components/modules illustrated. The examples disclosed herein may be described in the general context of computer code or machine-useable instructions, including computer-executable instructions such as program components, being executed by a computer or other machine, such as a personal data assistant or other handheld device. Generally, program components including routines, programs, objects, components, data structures, and the like, refer to code that performs particular tasks, or implement particular abstract data types. The disclosed examples may be practiced in a variety of system configurations, including personal computers, laptops, smart phones, mobile tablets, hand-held devices, consumer electronics, specialty computing devices, etc. The disclosed examples may also be practiced in distributed computing environments when tasks are performed by remote-processing devices that are linked through a communications network.

Computing device 900 includes a bus 920 that directly or indirectly couples the following devices: computer-storage memory 902, one or more processors 908, one or more presentation components 910, I/O ports 914, I/O components 916, a power supply 918, and a network component 912. While computing device 900 is depicted as a seemingly single device, multiple computing devices 900 may work together and share the depicted device resources. For example, memory 902 may be distributed across multiple devices, and processor(s) 908 may be housed with different devices.

Bus 920 represents what may be one or more busses (such as an address bus, data bus, or a combination thereof). Although the various blocks of FIG. 9 are shown with lines for the sake of clarity, delineating various components may be accomplished with alternative representations. For example, a presentation component such as a display device is an I/O component in some examples, and some examples of processors have their own memory. Distinction is not made between such categories as “workstation,” “server,” “laptop,” “hand-held device,” etc., as all are contemplated within the scope of FIG. 9 and the references herein to a “computing device.” Memory 902 may take the form of the computer-storage media references below and operatively provide storage of computer-readable instructions, data structures, program modules and other data for computing device 900. In some examples, memory 902 stores one or more of an operating system, a universal application platform, or other program modules and program data. Memory 902 is thus able to store and access data 904 and instructions 906 that are executable by processor 908 and configured to carry out the various operations disclosed herein.

In some examples, memory 902 includes computer-storage media in the form of volatile and/or nonvolatile memory, removable or non-removable memory, data disks in virtual environments, or a combination thereof. Memory 902 may include any quantity of memory associated with or accessible by computing device 900. Memory 902 may be internal to computing device 900 (as shown in FIG. 9), external to computing device 900, or both. Examples of memory 902 in include, without limitation, random access memory (RAM); read only memory (ROM); electronically erasable programmable read only memory (EEPROM); flash memory or other memory technologies; CD-ROM, digital versatile disks (DVDs) or other optical or holographic media; magnetic cassettes, magnetic tape, magnetic disk storage or other magnetic storage devices; memory wired into an analog computing device; or any other medium for encoding desired information and for access by computing device 900. Additionally, or alternatively, memory 902 may be distributed across multiple computing devices 900, for example, in a virtualized environment in which instruction processing is carried out on multiple computing devices 900. For the purposes of this disclosure, “computer storage media,” “computer-storage memory,” “memory,” and “memory devices” are synonymous terms for computer-storage memory 902, and none of these terms include carrier waves or propagating signaling.

Processor(s) 908 may include any quantity of processing units that read data from various entities, such as memory 902 or I/O components 916 and may include CPUs and/or GPUs. Specifically, processor(s) 908 are programmed to execute computer-executable instructions for implementing aspects of the disclosure. The instructions may be performed by the processor, by multiple processors within computing device 900, or by a processor external to client computing device 900. In some examples, processor(s) 908 are programmed to execute instructions such as those illustrated in the in the accompanying drawings. Moreover, in some examples, processor(s) 908 represent an implementation of analog techniques to perform the operations described herein. For example, the operations may be performed by an analog client computing device 900 and/or a digital client computing device 900. Presentation component(s) 910 present data indications to a user or other device. Exemplary presentation components include a display device, speaker, printing component, vibrating component, etc. One skilled in the art will understand and appreciate that computer data may be presented in a number of ways, such as visually in a graphical user interface (GUI), audibly through speakers, wirelessly between computing devices 900, across a wired connection, or in other ways. I/O ports 914 allow computing device 900 to be logically coupled to other devices including I/O components 916, some of which may be built in. Example I/O components 916 include, for example but without limitation, a microphone, joystick, game pad, satellite dish, scanner, printer, wireless device, etc.

Computing device 900 may operate in a networked environment via network component 912 using logical connections to one or more remote computers. In some examples, network component 912 includes a network interface card and/or computer-executable instructions (e.g., a driver) for operating the network interface card. Communication between computing device 900 and other devices may occur using any protocol or mechanism over any wired or wireless connection. In some examples, network component 912 is operable to communicate data over public, private, or hybrid (public and private) using a transfer protocol, between devices wirelessly using short range communication technologies (e.g., near-field communication (NFC), Bluetooth™ branded communications, or the like), or a combination thereof. Network component 912 communicates over wireless communication link 922 and/or a wired communication link 922 a to a cloud resource 924 across network 926. Various different examples of communication links 922 and 922a include a wireless connection, a wired connection, and/or a dedicated link, and in some examples, at least a portion is routed through the internet.

Although described in connection with an example computing device 900, examples of the disclosure are capable of implementation with numerous other general-purpose or special-purpose computing system environments, configurations, or devices. Examples of well-known computing systems, environments, and/or configurations that may be suitable for use with aspects of the disclosure include, but are not limited to, smart phones, mobile tablets, mobile computing devices, personal computers, server computers, hand-held or laptop devices, multiprocessor systems, gaming consoles, microprocessor-based systems, set top boxes, programmable consumer electronics, mobile telephones, mobile computing and/or communication devices in wearable or accessory form factors (e.g., watches, glasses, headsets, or earphones), network PCs, minicomputers, mainframe computers, distributed computing environments that include any of the above systems or devices, virtual reality (VR) devices, augmented reality (AR) devices, mixed reality devices, holographic device, and the like. Such systems or devices may accept input from the user in any way, including from input devices such as a keyboard or pointing device, via gesture input, proximity input (such as by hovering), and/or via voice input.

Examples of the disclosure may be described in the general context of computer-executable instructions, such as program modules, executed by one or more computers or other devices in software, firmware, hardware, or a combination thereof. The computer-executable instructions may be organized into one or more computer-executable components or modules. Generally, program modules include, but are not limited to, routines, programs, objects, components, and data structures that perform particular tasks or implement particular abstract data types. Aspects of the disclosure may be implemented with any number and organization of such components or modules. For example, aspects of the disclosure are not limited to the specific computer-executable instructions or the specific components or modules illustrated in the figures and described herein. Other examples of the disclosure may include different computer-executable instructions or components having more or less functionality than illustrated and described herein. In examples involving a general-purpose computer, aspects of the disclosure transform the general-purpose computer into a special-purpose computing device when configured to execute the instructions described herein.

By way of example and not limitation, computer readable media comprise computer storage media and communication media. Computer storage media include volatile and nonvolatile, removable and non-removable memory implemented in any method or technology for storage of information such as computer readable instructions, data structures, program modules, or the like. Computer storage media are tangible and mutually exclusive to communication media. Computer storage media are implemented in hardware and are non-transitory, i.e., exclude carrier waves and propagated signals. Computer storage media for purposes of this disclosure are not signals per se. Exemplary computer storage media include hard disks, flash drives, solid-state memory, phase change random-access memory (PRAM), static random-access memory (SRAM), dynamic random-access memory (DRAM), other types of random-access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transmission medium that may be used to store information for access by a computing device. In contrast, communication media typically embody computer readable instructions, data structures, program modules, or the like in a modulated data signal such as a carrier wave or other transport mechanism and include any information delivery media.

In some examples, a computer-implemented method includes generating a plurality of clusters; generating, for a new user, a profile; associating the generated profile into a cluster of the plurality of clusters; and generating, by a machine learning (ML) model, a recommendation for the user based on the associated cluster.

In some examples, an apparatus includes a user interface (UI); a memory; an image capturing device configured to capture a facial scan of a user; and a processor coupled to the memory configured to: control the UI to present a questionnaire; receive, via the UI, a response to the questionnaire; generate a profile associated with the user based on the received responses to the questionnaire and the captured facial scan; associate the generated profile into a cluster of a plurality of clusters; and execute, a machine learning (ML) model to generate a recommendation for the user based on the associated cluster.

In some examples, a computer-readable storage media stores instructions that, when executed by a processor, cause the processor to generate a plurality of clusters, each cluster of the generated plurality of clusters including at least a set of variables for users included in the cluster, the set related to at least one of gender, age, skin tone, acne marks, acne frequency, a lesion score, or a body distribution score; generate, for a new user, a profile, the generated profile including at least one variable of the set of variables; associate the generated profile into a cluster of the plurality of clusters; and executing a machine learning (ML) model to generate a recommendation for the user based on the associated cluster.

Further examples for generating a recommendation for a user are described herein.

Various examples further include one or more of the following:
wherein generating the plurality of clusters further comprises: identifying a plurality of existing profiles, each existing profiles of the plurality of existing profiles including a set of variables; for each of the existing profiles, identifying a value for each variable of the set of variables; and determining, by a clustering algorithm, a first existing profile and a second existing profile, of the plurality of existing profiles, have a similarity above a similarity threshold; and generating, by the clustering algorithm, the cluster including the first existing profile and the second existing profile;
wherein associating generated profile into the cluster of the plurality of clusters further comprises: identifying a set of variables for the generated profile; identifying a value for each variable of the set of variables for the generated profile; based on the identified value for each variable of the set of variables for the generated profile, determining, by the clustering algorithm, the cluster of the plurality of clusters most similar to the generated profile; and associating the generated profile into the determined cluster;
wherein the set of variables include variables related to at least one of gender, age, skin tone, acne marks, acne frequency, a lesion score, or a body distribution score;
wherein generating the recommendation for the user further comprises: determining a health outcome associated with the cluster of the plurality of clusters; identifying an intervention that, when applied, has a likelihood of addressing the determined health outcome; and generating, by the ML model, the recommendation for the user, the recommendation including the intervention;
wherein: the determined health outcome is acne; and the identified intervention is an acne treatment;
further comprising: receiving feedback indicating a result of the recommendation; and based on the received feedback, updating the ML model;
further comprising: receiving updated information from the new user; based on the received updated information, associating the generated profile into a second cluster of the plurality of clusters, the second cluster different than the cluster; and generating, by the ML model, a second recommendation for the user based on the associated second cluster;
further comprising: capturing a facial scan of the new user; and associating the generated profile into the cluster of the plurality of clusters based at least in part on the captured facial scan; and
wherein the recommendation is a recommendation related to acne.

The order of execution or performance of the operations in examples of the disclosure illustrated and described herein is not essential, and may be performed in different sequential manners in various examples. For example, it is contemplated that executing or performing a particular operation before, contemporaneously with, or after another operation is within the scope of aspects of the disclosure. When introducing elements of aspects of the disclosure or the examples thereof, the articles “a,” “an,” “the,” and “said” are intended to mean that there are one or more of the elements. The terms “comprising,” “including,” and “having” are intended to be inclusive and mean that there may be additional elements other than the listed elements. The term “exemplary” is intended to mean “an example of.” The phrase “one or more of the following: A, B, and C” means “at least one of A and/or at least one of B and/or at least one of C.”

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

1. A computer-implemented method, comprising:
generating a plurality of clusters;
generating, for a new user, a profile;
associating the generated profile into a cluster of the plurality of clusters; and
generating, by a machine learning (ML) model, a recommendation for the user based on the associated cluster.

2. The computer-implemented method of claim 1 wherein generating the plurality of clusters further comprises:
identifying a plurality of existing profiles, each existing profiles of the plurality of existing profiles including a set of variables;
for each of the existing profiles, identifying a value for each variable of the set of variables; and
determining, by a clustering algorithm, a first existing profile and a second existing profile, of the plurality of existing profiles, have a similarity above a similarity threshold; and
generating, by the clustering algorithm, the cluster including the first existing profile and the second existing profile.

3. The computer-implemented method of claim 2, wherein associating the generated profile into the cluster of the plurality of clusters further comprises:
identifying a set of variables for the generated profile;
identifying a value for each variable of the set of variables for the generated profile;
based on the identified value for each variable of the set of variables for the generated profile, determining, by the clustering algorithm, the cluster of the plurality of clusters most similar to the generated profile; and
associating the generated profile into the determined cluster.

4. The computer-implemented method of claim 3, wherein the set of variables include variables related to at least one of gender, age, skin tone, acne marks, acne frequency, a lesion score, or a body distribution score.

5. The computer-implemented method of claim 1, wherein generating the recommendation for the user further comprises:
determining a health outcome associated with the cluster of the plurality of clusters;
identifying an intervention that, when applied, has a likelihood of addressing the determined health outcome; and
generating, by the ML model, the recommendation for the user, the recommendation including the intervention.

6. The computer-implemented method of claim 5, wherein:
the determined health outcome is acne; and
the identified intervention is an acne treatment.

7. The computer-implemented method of claim 1, further comprising:
receiving feedback indicating a result of the recommendation; and
based on the received feedback, updating the ML model.

8. The computer-implemented method of claim 1, further comprising:
receiving updated information from the new user;
based on the received updated information, associating the generated profile into a second cluster of the plurality of clusters, the second cluster different than the cluster; and
generating, by the ML model, a second recommendation for the user based on the associated second cluster.

9. The computer-implemented method of claim 1, further comprising:
capturing a facial scan of the new user; and
associating the generated profile into the cluster of the plurality of clusters based at least in part on the captured facial scan.

10. The computer-implemented method of claim 1, wherein the recommendation is a recommendation related to an acne treatment regimen.

11. An apparatus comprising:
a user interface (UI);
a memory;
an image capturing device configured to capture a facial scan of a user; and
a processor coupled to the memory configured to:
control the UI to present a questionnaire;
receive, via the UI, a response to the questionnaire;
generate a profile associated with the user based on the received responses to the questionnaire and the captured facial scan;
associate the generated profile into a cluster of a plurality of clusters; and
execute a machine learning (ML) model to generate a recommendation for the user based on the associated cluster.

12. The apparatus of claim 11, wherein the processor is further configured to:
identify a plurality of existing profiles, each existing profile of the plurality of existing profiles including a set of variables;
for each of the existing profiles, identify a value for each variable of the set of variables; and
execute a clustering algorithm to determine a first existing profile and a second existing profile, of the plurality of existing profiles, have a similarity above a similarity threshold; and
generate, by the clustering algorithm, the cluster including the first existing profile and the second existing profile.

13. The apparatus of claim 12, wherein, to associate the generated profile into the cluster of the plurality of clusters, the processor is further configured to:
identify a set of variables for the generated profile;
identify a value for each variable of the set of variables for the generated profile;
based on the identified value for each variable of the set of variables for the generated profile, executed the clustering algorithm to determine the cluster of the plurality of clusters most similar to the generated profile; and
associate the generated profile into the determined cluster.

14. The apparatus of claim 13, wherein the set of variables include variables related to at least one of gender, age, skin tone, acne marks, acne frequency, a lesion score, or a body distribution score.

15. The apparatus of claim 11, wherein, to generate the recommendation for the user, the processor is further configured to:
determine a health outcome associated with the cluster of the plurality of clusters;
identify an intervention that, when applied, has a likelihood of addressing the determined health outcome; and
execute the ML model to generate the recommendation for the user, the recommendation including the intervention.

16. The apparatus of claim 11, wherein the processor is further configured to:
receive updated information from the new user;
based on the received updated information, associate the generated profile into a second cluster of the plurality of clusters, the second cluster different than the cluster; and
execute the ML model to generate a second recommendation for the user based on the associated second cluster.

17. The apparatus of claim 11, wherein the processor is further configured to:
capture a facial scan of the new user; and
associate the generated profile into the cluster of the plurality of clusters based at least in part on the captured facial scan.

18. One or more non-transitory computer readable media storing instructions that, when executed by a processor, cause the processor to:
generate a plurality of clusters, each cluster of the generated plurality of clusters including at least a set of variables for users included in the cluster, the set of variables related to at least one of gender, age, skin tone, acne marks, acne frequency, a lesion score, or a body distribution score;
generate, for a new user, a profile, the generated profile including at least one variable of the set of variables;
associate the generated profile into a cluster of the plurality of clusters; and
executing a machine learning (ML) model to generate a recommendation for the user based on the associated cluster.

19. The one or more non-transitory computer readable media of claim 18, further storing instructions for generating the recommendation for the user that, when executed by the processor, cause the processor to:
determine a health outcome associated with the cluster of the plurality of clusters, the determined health outcome including an amount or type of acne;
identify an intervention associated with the associated cluster that, when applied, has a likelihood of addressing the determined health outcome, the identified intervention including an acne treatment; and
execute the ML model to generate the recommendation for the user, the recommendation including the intervention.

20. The one or more non-transitory computer readable media of claim 18, further storing instructions for generating the recommendation for the user that, when executed by the processor, cause the processor to:
receive updated information from the new user;
based on the received updated information, associate the generated profile into a second cluster of the plurality of clusters, the second cluster different than the cluster; and
execute the ML model to generate a second recommendation for the user based on the associated second cluster.
